(19) United States
(12) Patent Application Publication
(10) Pub. No.: US 2026/0293857 A1
(43) Pub. Date: Oct. 1, 2026

(54) FEED INTAKE MONITORING DEVICE AND SYSTEM BASED ON PRESSURE SENSING DURING CATTLE JAW MOVEMENT

(71) Applicant: Information Technology Research Center, Beijing Academy of Agriculture and Forestry Sciences, BEIJING (CN)

(72) Inventors: Luyu DING, Beijing (CN); Qifeng LI, Beijing (CN); Zhuo LI, Beijing (CN); Weihong MA, Beijing (CN); Ligen YU, Beijing (CN); Rui MENG, Beijing (CN); Qinyang YU, Beijing (CN); Ronghua GAO, Beijing (CN)

(21) Appl. No.: 19/571,841

(22) Filed: Mar. 19, 2026

(30) Foreign Application Priority Data
Mar. 25, 2025 (CN) 202510356113.3

(51) Int. Cl. A01K 29/00

(52) U.S. Cl. CPC A01K 29/005; A01K 2227/101

(57) ABSTRACT

Provided are a feed intake monitoring device and system for cattle based on pressure sensing during jaw movement. The device includes a main control module, a liquid sac, and a fastening strap which can be worn on cattle head. The liquid sac is attached to the fastening strap, with its lower surface fitting against an upper portion of a nose ring. The main control module is arranged over the liquid sac and includes a microcontroller unit, a liquid pressure sensor, and a communication module. The liquid pressure sensor acquires pressure data of the liquid sac during jaw movement. The microcontroller unit is configured to transmit the data to an application platform via the communication module. The system includes: a feed intake monitoring device mentioned above; and the application platform, which analyzes the pressure data and output or show the individual feed intake of the cattle.

### CROSS REFERENCE TO RELATED APPLICATION

This patent application claims the benefit and priority of Chinese Patent Application No. 2025103561133, filed with the China National Intellectual Property Administration on Mar. 25, 2025, the disclosure of which is incorporated by reference herein in its entirety as part of the present application.

### TECHNICAL FIELD

The present disclosure relates to the technical field of intelligent livestock farming, and in particular, to a feed intake monitoring device and system based on pressure sensing during cattle jaw movement.

### BACKGROUND

Feed intake is a direct indicator reflecting the nutritional status of livestock. Monitoring the feed intake of cattle not only can help farmers optimize feeding plans and reduce feed waste, but also enables timely detection and treatment of diseases, ensuring the health of the cattle. Currently, methods for determining feed intake both domestically and internationally are mainly divided into two categories: plant-based methods and animal-based methods. Plant-based methods are primarily applied to grazing cattle, where a feed intake is determined by measuring changes in grassland plant biomass before and after grazing. Such methods are also referred to as a difference method. Animal-based methods mainly include a grazing behavior method, a method based on a relationship between digestibility and fecal output, an alkane method, a near-infrared method, and a feed weighing trough method, among others.

The grazing behavior method estimates a feed intake through regression according to cattle behaviors such as feeding, rumination, and chewing and characterization parameters thereof. Under traditional conditions, this method relies on manual observation of behaviors. Subsequently, it has evolved to monitor cattle behaviors through two approaches: image recognition and wearable devices, e.g., those based on image sensors, sound sensors, and inertial sensors. However, the use of the image sensors requires substantial storage space; the sound sensors are susceptible to noise interference; and the inertial sensors are prone to head movement disturbances, such as shaking or nodding.

### SUMMARY

The present disclosure provides a feed intake monitoring device and system based on pressure sensing during cattle jaw movement to overcome the drawbacks in the prior art such as requiring substantial storage space, susceptibility to noise interference, and susceptibility to head movement disturbances, achieving the effects of reducing the demand for storage space, avoiding noise interference, and avoiding head movement disturbances.

The present disclosure provides a feed intake monitoring device based on pressure sensing during cattle jaw movement, including a main control module, a liquid sac, and a fastening strap, where:
the fastening strap is configured to be worn on the head of cattle;
the liquid sac is attached to the fastening strap, and upon wearing, a lower surface of the liquid sac fits against an upper portion of a nose ring;
the main control module is arranged over the liquid sac and includes a microcontroller unit, a liquid pressure sensor, and a communication module;
the liquid pressure sensor is configured to acquire pressure data of the liquid sac during cattle jaw movement;
the microcontroller unit is configured to transmit the pressure data to an application platform via the communication module; and the application platform is configured to analyze the pressure data and output an individual feed intake of the cattle.

According to the feed intake monitoring device based on pressure sensing during cattle jaw movement provided by the present disclosure, the liquid sac is made up of an upper layer and a lower layer that are bonded together in an arc shape to fit against the upper portion of the nose ring, where the upper layer is a flat layer, and the lower layer is recessed downward; a cavity between the upper layer and the lower layer is filled with a liquid medium; and a snap-in connection is achieved using a snap-in structure at a junction of the upper layer and the lower layer.

According to the feed intake monitoring device based on pressure sensing during cattle jaw movement provided by the present disclosure, the liquid medium may be propylene glycol.

According to the feed intake monitoring device based on pressure sensing during cattle jaw movement provided by the present disclosure, the main control module may be provided with a liquid replenishing opening that communicates with an interior of the liquid sac and is configured to replenish the liquid sac with the liquid medium.

According to the feed intake monitoring device based on pressure sensing during cattle jaw movement provided by the present disclosure, the feed intake monitoring device based on pressure sensing during cattle jaw movement further includes a lithium battery, where the main control module further includes a power module;
the lithium battery is configured to supply power to the microcontroller unit; and
the power module has a charging interface for charging the lithium battery.

According to the feed intake monitoring device based on pressure sensing during cattle jaw movement provided by the present disclosure, the main control module further includes a storage module;
the microcontroller unit is further configured to compress the pressure data to obtain a compressed file;
the storage module is configured to store the compressed file; and
the microcontroller unit is further configured to transmit the stored compressed file to the application platform via the communication module.

According to the feed intake monitoring device based on pressure sensing during cattle jaw movement provided by the present disclosure, the main control module is provided with a switch button that is designed to be recessed and concealed.

According to the feed intake monitoring device based on pressure sensing during cattle jaw movement provided by the present disclosure, when the pressure reaches a threshold, the liquid pressure sensor is triggered to acquire the pressure data with a sampling frequency of 2-5 Hz;
in an operating state, the feed intake monitoring device based on pressure sensing during cattle jaw movement includes a low-power mode and a high-performance mode; when data acquisition and data transmission are not triggered, the feed intake monitoring device based on pressure sensing during cattle jaw movement is in the low-power mode; and when data acquisition and data transmission are triggered, the feed intake monitoring device based on pressure sensing during cattle jaw movement is in the high-performance mode.

The present disclosure further provides a feed intake monitoring system based on pressure sensing during cattle jaw movement, including an application platform and the feed intake monitoring device based on pressure sensing during cattle jaw movement as described above, where:
the feed intake monitoring device based on pressure sensing during cattle jaw movement is configured to acquire the pressure data of the liquid sac during the cattle jaw movement and transmit the pressure data to the application platform; and
the application platform is configured to analyze the pressure data so as to obtain an individual feed intake of the cattle, and output and show the individual feed intake.

According to the feed intake monitoring system based on pressure sensing during cattle jaw movement provided by the present disclosure, the application platform is further configured to identify, according to the pressure data, a rate and a duration of a feeding behavior, and a duration and a chewing frequency of a ruminating behavior, estimate a feed intake of the cattle according to the rate and the duration of the feeding behavior, and calibrate the estimated feeding intake according to the duration and the chewing frequency of the ruminating behavior.

The present disclosure provides the feed intake monitoring device and system based on pressure sensing during cattle jaw movement. The feed intake monitoring device based on pressure sensing during cattle jaw movement is worn on the head of the cattle by using the fastening strap. The liquid sac is attached to the fastening strap, and upon wearing, the lower surface of the liquid sac fits against the upper portion of the nose ring. The pressure data of the liquid sac during the cattle jaw movement is acquired by the liquid pressure sensor. The pressure data is then transmitted to the application platform by the microcontroller unit via the communication module. The pressure data is analyzed by the application platform, and the individual feed intake of the cattle is output. The problems of the demand for substantial storage space when using image sensors, the susceptibility to noise interference when using sound sensors, and the susceptibility to head movement disturbances when using inertial sensors are avoided, achieving the effects of reducing the demand for storage space, avoiding the noise interference, and avoiding the head movement disturbances.

### BRIEF DESCRIPTION OF THE DRAWINGS

To describe the technical solutions in the embodiments of the present disclosure or in the prior art more clearly, the following briefly describes the accompanying drawings required for describing the embodiments or the prior art. Apparently, the accompanying drawings in the following description show some embodiments of the present disclosure, and a person skilled in the art may still derive other drawings from these accompanying drawings without creative efforts.

FIG. 1 is a first schematic structural diagram of a feed intake monitoring device based on pressure sensing during cattle jaw movement provided by the present disclosure;

FIG. 2 is a schematic diagram of an outline of a fastening strap of a feed intake monitoring device based on pressure sensing during cattle jaw movement provided by the present disclosure;

FIG. 3 is a schematic diagram of a feed intake monitoring device based on pressure sensing during cattle jaw movement provided by the present disclosure being worn on the mouth of cattle;

FIG. 4 is a schematic structural diagram of a liquid sac of a feed intake monitoring device based on pressure sensing during cattle jaw movement provided by the present disclosure;

FIG. 5 is a schematic diagram of a snap-in structure at a junction of an upper layer and a lower layer of a liquid sac provided by the present disclosure;

FIG. 6 is a second schematic structural diagram of a feed intake monitoring device based on pressure sensing during cattle jaw movement provided by the present disclosure; and

FIG. 7 is an architecture diagram of a feed intake monitoring system based on pressure sensing during cattle jaw movement provided by the present disclosure.

### DETAILED DESCRIPTION OF THE EMBODIMENTS

To make the objective, technical solutions, and advantages of the present disclosure clearer, the technical solutions in the present disclosure will be described clearly and completely below with reference to the accompanying drawings in the present disclosure. Apparently, the described embodiments are part of rather than all of the embodiments of the present disclosure. All other embodiments derived from the embodiments of the present disclosure by a person of ordinary skill in the art without creative efforts shall fall within the protection scope of the present disclosure.

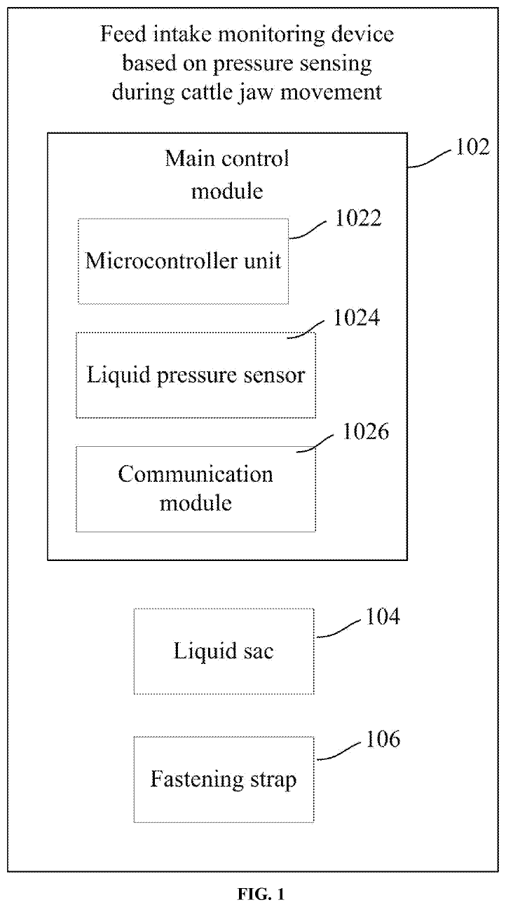

The feed intake monitoring device and system based on pressure sensing during cattle jaw movement provided by the present disclosure are described below in conjunction with FIG. 1 to FIG. 7.

FIG. 1 is a first schematic structural diagram of a feed intake monitoring device based on pressure sensing during cattle jaw movement provided by the present disclosure. As shown in FIG. 1, the device includes a main control module 102, a liquid sac 104, and a fastening strap 106. The main control module 102 includes a microcontroller unit 1022, a liquid pressure sensor 1024, and a communication module 1026.

The fastening strap 106 is configured to be worn on the head of cattle.

The liquid sac 104 is attached to the fastening strap, and upon wearing, a lower surface of the liquid sac fits against an upper portion of a nose ring.

The main control module 102 is arranged over the liquid sac.

The liquid pressure sensor 1024 is configured to acquire pressure data of the liquid sac during cattle jaw movement.

The microcontroller unit 1022 is configured to transmit the pressure data to an application platform via the communication module 1026. The application platform is configured to analyze the pressure data and output an individual feed intake of the cattle.

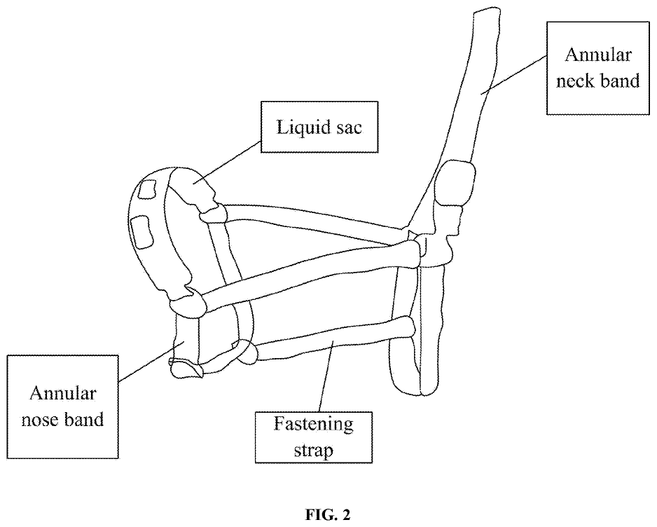

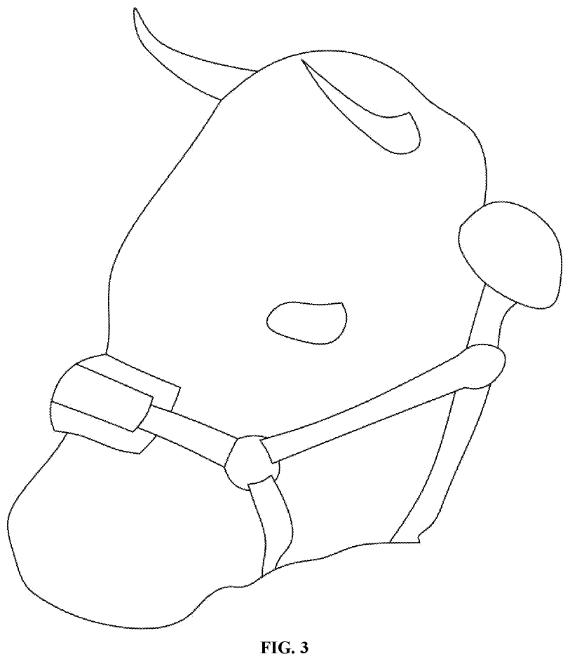

The fastening strap is configured to attach a core component of the device and is worn on the head of the cattle. As shown in FIG. 2, the fastening strap includes an annular neck band and an annular nose band that are configured to be annularly attached onto the neck and nose of the cattle, respectively. The annular neck band and the annular nose band are elastically adjustable and may be fastened using buckles and connected end to end. The fastening strap is designed to be elastically adjustable to adapt to cattle of different bodily forms. The fastening strap may be added with a non-slip design to prevent the device from sliding or falling off. The fastening strap may be made of a flexible material, guaranteeing securing while not hurting the cattle. The fastening strap is attached with the liquid sac, the main control module, a main control module housing, and a lithium battery. The wearing effect is as shown in FIG. 3.

The liquid sac is made up of an upper layer and a lower layer that are bonded together. The upper layer of the liquid sac fits against and is attached to the fastening strap without bearing the tensile force of the fastening strap, while its lower surface is designed to fit against an upper portion of a nose ring upon wearing. The material of the liquid sac may be selected from flexible materials that are stretch-resistant, abrasion-resistant, and waterproof to minimize wear and the risk of liquid leakage. For example, thermoplastic polyurethane (TPU) may be chosen.

The liquid pressure sensor is designed with an inter-integrated circuit (IIC) communication circuit to connect with the microcontroller unit, and has a measurement range of 1000-1500 mBar, a sensor accuracy of 100-200 mBar, and a sampling frequency of 2-5 Hz. A trigger threshold is set for pressure data acquisition to reduce data load. The acquisition of the pressure data begins when the pressure data reaches the threshold (e.g., 800 mBar), thereby avoiding invalid data acquisition.

The communication module may be a 4G CAT1 module. The communication module may be powered by the microcontroller unit. The communication module may be designed with an antenna interface to optimize communication quality. The communication module may be integrated with a universal serial bus (USB) interface and a universal subscriber identity module (USIM) card slot for inserting a Micro SIM card.

The main control module housing is arranged outside the main control module. The main control module housing is an impact-resistant housing for protecting the internal circuitry and the sensor. It is made of a weather-resistant material suitable for outdoor environments, with a waterproof rating of IP65/IP67, effectively preventing damage from rainwater and cattle licking.

The present disclosure provides the feed intake monitoring device based on pressure sensing during cattle jaw movement. The feed intake monitoring device based on pressure sensing during cattle jaw movement is worn on the head of the cattle by using the fastening strap. The liquid sac is attached to the fastening strap, and upon wearing, the lower surface of the liquid sac fits against the upper portion of the nose ring. The pressure data of the liquid sac during the cattle jaw movement is acquired by the liquid pressure sensor. The pressure data is then transmitted to the application platform by the microcontroller unit via the communication module. The pressure data is analyzed by the application platform, and the individual feed intake of the cattle is output. The problems of the demand for substantial storage space when using image sensors, the susceptibility to noise interference when using sound sensors, and the susceptibility to head movement disturbances when using inertial sensors are avoided, achieving the effects of reducing the demand for storage space, avoiding the noise interference, and avoiding the head movement disturbances. Furthermore, by transmitting the pressure data to the application platform for analysis, the problem of cumbersome data usage caused by relying solely on local data storage on the device is avoided, thereby enhancing the efficiency of data processing and analysis. The use of the liquid sac enables rapid detection of changes in pressure value, and the pressure distribution is uniform. During the cattle jaw movement, the pressure data can be promptly captured, ensuring stable operation of the device while improving the sampling accuracy of the pressure data, thereby providing more accurate data. The liquid sac is attached to the fastening strap without bearing the tensile force of the fastening strap, thereby avoiding the problem of stretching fatigue of the liquid sac due to stretching stress. The present disclosure is applicable to cattle under both confined housing and grazing conditions. By wearing the feed intake monitoring device based on pressure sensing during cattle jaw movement on cattle, real-time monitoring of the pressure data of the cattle's mouth can be achieved. On this basis, the number of behaviors and the feed intake can be calculated, assisting farming personnel in evaluating the cattle's feed consumption and feed utilization efficiency. Thus, feeding plans can be adjusted, assisting in monitoring cattle health conditions and providing new technical equipment for scientific and accurate production and husbandry practices.

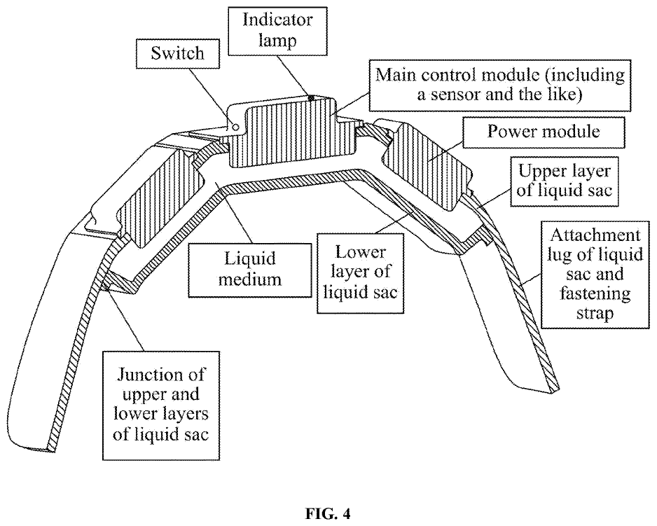

In an embodiment, as shown in FIG. 4, the liquid sac is made up of an upper layer and a lower layer that are bonded together in an arc shape to fit against the upper portion of the nose ring, where the upper layer is a flat layer, and the lower layer is recessed downward; a cavity between the upper layer and the lower layer is filled with a liquid medium; and a snap-in connection is achieved using a snap-in structure at a junction of the upper layer and the lower layer.

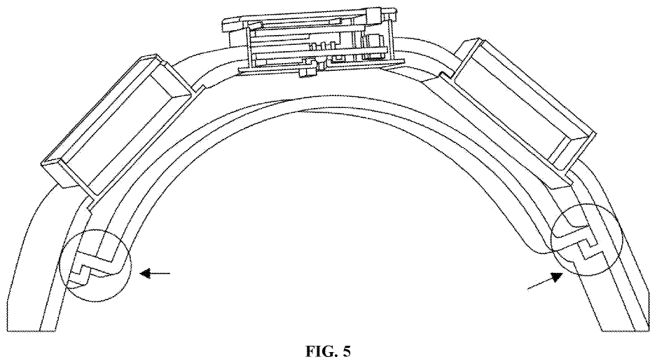

The snap-in structure may be a C-shaped snap-in structure. The parts circled out and indicated by the arrows in FIG. 5 are C-shaped snap-in structures at the junctions of the upper and lower layers.

In the above embodiment, the snap-in connection is achieved using the snap-in structure at the junction of the upper layer and the lower layer of the liquid sac, facilitating bonding and avoiding the risk of liquid leakage. The entire part is designed in the arc shape to fit against the upper portion of the nose ring, ensuring wearing comfort.

In an embodiment, the liquid medium includes propylene glycol.

In the above embodiment, the liquid medium filled in the liquid sac includes propylene glycol, which is non-toxic, pollution-free, and low-cost and can ensure that the pressure sensing signal remains virtually unaffected by high or low temperature conditions and the pressure applied is stable.

In an embodiment, the main control module is provided with a liquid replenishing opening that communicates with an interior of the liquid sac and is configured to replenish the liquid sac with the liquid medium.

A sealing screw may be disposed on the liquid replenishing opening. After the sealing screw is unscrewed, the liquid may be replenished through the liquid replenishing opening.

In the above embodiment, by providing the liquid replenishing opening in the main control module, replenishing the liquid sac with the liquid is achieved, avoiding the problem that the liquid cannot be replenished.

In an embodiment, the feed intake monitoring device based on pressure sensing during cattle jaw movement further includes a lithium battery. The main control module further includes a power module. The lithium battery is configured to supply power to the microcontroller unit. The power module has a charging interface for charging the lithium battery.

The power module includes an electric quantity monitoring chip for power management. An overcharging and overdischarging protection circuit is designed, and a power indicator lamp is designed for checking the power state.

In the above embodiment, the rechargeable lithium battery is employed to supply power, which is convenient to use. The power module has the charging interface, so that the lithium battery can be charged without being removed. Thus, the troublesome operations of wearing and taking off the device repeatedly are reduced.

In an embodiment, the main control module further includes a storage module. The microcontroller unit is further configured to compress the pressure data to obtain a compressed file. The storage module is configured to store the compressed file. The microcontroller unit is further configured to transmit the stored compressed file to the application platform via the communication module.

The storage module includes a secure digital (SD) memory drive to ensure sufficient storage space for data spanning a target number of days, for example, enough for 7 days of storage. An SD multi-media card (SDMMC) interface is designed to connect the microcontroller unit and the SD card, providing file allocation table file system (FATFS) support for storing compressed files. The stored compressed files may be transmitted to the application platform via the communication module at a first preset time intervals, achieving intermittent transmission.

In the above embodiment, the pressure data is compressed to obtain the compressed file. The compressed file is transmitted to the application platform. Thus, the data transmission time can be shortened and the transmission efficiency can be improved, reducing problems such as data backlog and excessive transmission time caused by large data volumes. The compressed file is stored in the storage module, and the stored compressed file is then transmitted, so that data loss can be avoided.

In an embodiment, the main control module is provided with a switch button that is designed to be recessed and concealed.

The switch button is configured to control the start, stop, and reset of data acquisition by the feed intake monitoring device based on pressure sensing during cattle jaw movement.

In the above embodiment, the use of a concealed switch design reduces the risk of accidental activation and enhances aesthetic appearance.

In an embodiment, when the pressure reaches a threshold, the liquid pressure sensor is triggered to acquire the pressure data with a sampling frequency of 2-5 Hz. In an operating state, the feed intake monitoring device based on pressure sensing during cattle jaw movement includes a low-power mode and a high-performance mode. When data acquisition and data transmission are not triggered, the feed intake monitoring device based on pressure sensing during cattle jaw movement is in the low-power mode; and when data acquisition and data transmission are triggered, the feed intake monitoring device based on pressure sensing during cattle jaw movement is in the high-performance mode.

After power is supplied and the switch is turned on, the device enters its operating state, and in the operating state, includes the low-power mode and the high-performance mode.

In the above embodiment, when the pressure reaches the threshold, the liquid pressure sensor is triggered to acquire the pressure data with the sampling frequency of 2-5 Hz. Thus, the data load can be reduced. When data acquisition and data transmission are not triggered, the feed intake monitoring device based on pressure sensing during cattle jaw movement is in the low-power mode; and when data acquisition and data transmission are triggered, the feed intake monitoring device based on pressure sensing during cattle jaw movement is in the high-performance mode. Thus, the power consumption can be reduced.

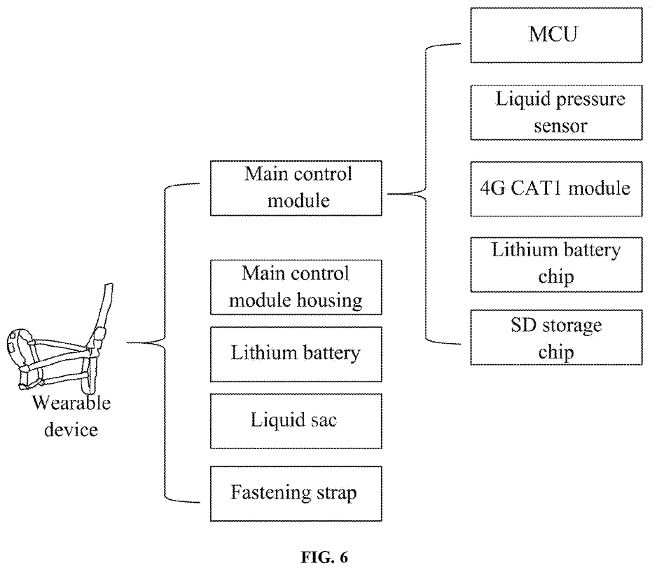

In an embodiment, as shown in FIG. 6, the feed intake monitoring device based on pressure sensing during cattle jaw movement (also referred to as a wearable device) includes a main control module, a main control module housing, a lithium battery, a liquid sac, and a fastening strap. The main control module includes a microcontroller unit (MCU), a liquid pressure sensor, a communication module (e.g., a 4G CAT1 module), a power module (e.g., a lithium battery chip), and a storage module (e.g., an SD storage chip). The microcontroller unit is powered by the lithium battery. The liquid pressure sensor, the communication module, the lithium battery chip, and the SD storage chip are connected to the MCU for supplying power.

The feed intake monitoring system based on pressure sensing during cattle jaw movement is described below. The feed intake monitoring system based on pressure sensing during cattle jaw movement described below and the feed intake monitoring device based on pressure sensing during cattle jaw movement described above can be cross-referenced.

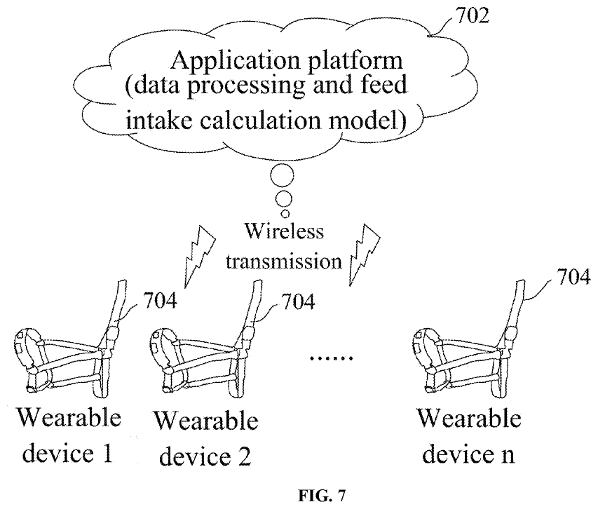

As shown in FIG. 7, the present disclosure provides a feed intake monitoring system based on pressure sensing during cattle jaw movement, including an application platform 702 and a feed intake monitoring device 704 based on pressure sensing during cattle jaw movement.

The feed intake monitoring device 704 based on pressure sensing during cattle jaw movement is configured to acquire pressure data of a liquid sac during cattle jaw movement and transmit the pressure data to the application platform.

The application platform 702 is configured to analyze the pressure data so as to obtain an individual feed intake of the cattle, and output and show the individual feed intake.

The feed intake monitoring device based on pressure sensing during cattle jaw movement may compress the pressure data to obtain a compressed file and transmit the compressed file to the application platform. The application platform receives the compressed file and uncompresses it for storage in a database. The application platform is deployed with a feed intake calculation module. The data in the database is called, and the individual feed intake of the cattle is calculated by the feed intake calculation module at a second preset time interval, and output and shown. For example, the second preset time interval may be one hour.

In the feed intake monitoring system based on pressure sensing during cattle jaw movement described above, the feed intake monitoring device based on pressure sensing during cattle jaw movement acquires the pressure data of the liquid sac during the cattle jaw movement and transmits the pressure data to the application platform. The pressure data is analyzed by the application platform to obtain the individual feed intake of the cattle, and the individual feed intake is output and shown. The problems of the demand for substantial storage space when using image sensors, the susceptibility to noise interference when using sound sensors, and the susceptibility to head movement disturbances when using inertial sensors are avoided, achieving the effects of reducing the demand for storage space, avoiding the noise interference, and avoiding the head movement disturbances.

In an embodiment, the application platform is further configured to identify, according to the pressure data, a rate and a duration of a feeding behavior, and a duration and a chewing frequency of a ruminating behavior, estimate a feed intake of the cattle according to the rate and the duration of the feeding behavior, and calibrate the estimated feeding intake according to the duration and the chewing frequency of the ruminating behavior.

In the above embodiment, the application platform is employed to identify, according to the pressure data, the rate and the duration of the feeding behavior, and the duration and the chewing frequency of the ruminating behavior, estimate the feed intake of the cattle according to the rate and the duration of the feeding behavior, and calibrate the estimated feeding intake according to the duration and the chewing frequency of the ruminating behavior. Thus, the individual feed intake of the cattle can be determined accurately.

Finally, it should be noted that the foregoing examples are only used to illustrate the technical solutions of the present disclosure, and are not intended to limit the present disclosure. Although the present disclosure is described in detail with reference to the foregoing examples, those of ordinary skill in the art should understand that they can still modify the technical solutions described in the foregoing examples, or make equivalent substitutions to some technical features therein. These modifications or substitutions do not make the essence of the corresponding technical solutions depart from the spirit and scope of the technical solutions in the examples of the present disclosure.

What is claimed is:

1. A feed intake monitoring device based on pressure sensing during cattle jaw movement, comprising a main control module, a liquid sac, and a fastening strap, wherein
the fastening strap is configured to be worn on the head of cattle;
the liquid sac is attached to the fastening strap, and upon wearing, a lower surface of the liquid sac fits against an upper portion of a nose ring;
the main control module is arranged over the liquid sac and comprises a microcontroller unit, a liquid pressure sensor, and a communication module;
the liquid pressure sensor is configured to acquire pressure data of the liquid sac during cattle jaw movement;
the microcontroller unit is configured to transmit the pressure data to an application platform via the communication module, so that the application platform determines an individual feed intake of the cattle based on the pressure data.

2. The feed intake monitoring device based on pressure sensing during cattle jaw movement according to claim 1, wherein the liquid sac comprises an upper layer and a lower layer that are bonded together in an arc shape to fit against the upper portion of the nose ring, wherein the upper layer is a flat layer, and the lower layer is recessed downward; a cavity between the upper layer and the lower layer is filled with a liquid medium; and a snap-in connection is achieved using a snap-in structure at a junction of the upper layer and the lower layer.

3. The feed intake monitoring device based on pressure sensing during cattle jaw movement according to claim 2, wherein the liquid medium comprises propylene glycol.

4. The feed intake monitoring device based on pressure sensing during cattle jaw movement according to claim 1, wherein the main control module is provided with a liquid replenishing opening that communicates with an interior of the liquid sac and is configured to replenish the liquid sac with the liquid medium.

5. The feed intake monitoring device based on pressure sensing during cattle jaw movement according to claim 1, further comprising a lithium battery, wherein the main control module further comprises a power module;
the lithium battery is configured to supply power to the microcontroller unit; and
the power module has a charging interface for charging the lithium battery.

6. The feed intake monitoring device based on pressure sensing during cattle jaw movement according to claim 1, wherein the main control module further comprises a storage module;
the microcontroller unit is further configured to compress the pressure data to obtain a compressed file;
the storage module is configured to store the compressed file; and
the microcontroller unit is further configured to transmit the stored compressed file to the application platform via the communication module.

7. The feed intake monitoring device based on pressure sensing during cattle jaw movement according to claim 1, wherein the main control module is provided with a switch button that is designed to be recessed and concealed.

8. The feed intake monitoring device based on pressure sensing during cattle jaw movement according to claim 1, wherein when the pressure reaches a threshold, the liquid pressure sensor is triggered to acquire the pressure data with a sampling frequency of 2-5 Hz;
in an operating state, the feed intake monitoring device based on pressure sensing during cattle jaw movement comprises a low-power mode and a high-performance mode; when data acquisition and data transmission are not triggered, the feed intake monitoring device based on pressure sensing during cattle jaw movement is in the low-power mode; and when data acquisition and data transmission are triggered, the feed intake monitoring device based on pressure sensing during cattle jaw movement is in the high-performance mode.

9. A feed intake monitoring system based on pressure sensing during cattle jaw movement, comprising an application platform and the feed intake monitoring device based on pressure sensing during cattle jaw movement according to claim 1, wherein
the feed intake monitoring device based on pressure sensing during cattle jaw movement is configured to acquire the pressure data of the liquid sac during the cattle jaw movement and transmit the pressure data to the application platform; and
the application platform is configured to analyze the pressure data so as to obtain an individual feed intake of the cattle, and output and show the individual feed intake.

10. The feed intake monitoring system based on pressure sensing during cattle jaw movement according to claim 9, wherein the application platform is further configured to identify, according to the pressure data, a rate and a duration of a feeding behavior, and a duration and a chewing frequency of a ruminating behavior, estimate a feed intake of the cattle according to the rate and the duration of the feeding behavior, and calibrate the estimated feeding intake according to the duration and the chewing frequency of the ruminating behavior.

11. The feed intake monitoring system based on pressure sensing during cattle jaw movement according to claim 9, wherein the liquid sac comprises an upper layer and a lower layer that are bonded together in an arc shape to fit against the upper portion of the nose ring, wherein the upper layer is a flat layer, and the lower layer is recessed downward; a cavity between the upper layer and the lower layer is filled with a liquid medium; and a snap-in connection is achieved using a snap-in structure at a junction of the upper layer and the lower layer.

12. The feed intake monitoring system based on pressure sensing during cattle jaw movement according to claim 11, wherein the liquid medium comprises propylene glycol.

13. The feed intake monitoring system based on pressure sensing during cattle jaw movement according to claim 9, wherein the main control module is provided with a liquid replenishing opening that communicates with an interior of the liquid sac and is configured to replenish the liquid sac with the liquid medium.

14. The feed intake monitoring system based on pressure sensing during cattle jaw movement according to claim 9, further comprising a lithium battery, wherein the main control module further comprises a power module;
the lithium battery is configured to supply power to the microcontroller unit; and
the power module has a charging interface for charging the lithium battery.

15. The feed intake monitoring system based on pressure sensing during cattle jaw movement according to claim 9, wherein the main control module further comprises a storage module;
the microcontroller unit is further configured to compress the pressure data to obtain a compressed file;
the storage module is configured to store the compressed file; and
the microcontroller unit is further configured to transmit the stored compressed file to the application platform via the communication module.

16. The feed intake monitoring system based on pressure sensing during cattle jaw movement according to claim 9, wherein the main control module is provided with a switch button that is designed to be recessed and concealed.

17. The feed intake monitoring system based on pressure sensing during cattle jaw movement according to claim 9, wherein when the pressure reaches a threshold, the liquid pressure sensor is triggered to acquire the pressure data with a sampling frequency of 2-5 Hz;
in an operating state, the feed intake monitoring device based on pressure sensing during cattle jaw movement comprises a low-power mode and a high-performance mode; when data acquisition and data transmission are not triggered, the feed intake monitoring device based on pressure sensing during cattle jaw movement is in the low-power mode; and when data acquisition and data transmission are triggered, the feed intake monitoring device based on pressure sensing during cattle jaw movement is in the high-performance mode.
